(19) United States
(12) Patent Application Publication
(10) Pub. No.: US 2023/0374438 A1
(43) Pub. Date: Nov. 23, 2023

(54) SYSTEM FOR OBTAINING IMAGE OF A PLATED CULTURE DISH USING AN IMAGING DEVICE HAVING A TELECENTRIC LENS

(71) Applicant: BD KIESTRA B.V., Drachten (NL)

(72) Inventors: Raphael R. Marcelpoil, Corenc (FR); Roger Guido Petri, Assen (NL); Mathieu Julien Fernandes, Coublevie (FR); Johannes Wijnandus Thiecke, Groningen (NL)

(73) Assignee: BD KIESTRA B.V., Drachten (NL)

(21) Appl. No.: 18/030,123

(22) PCT Filed: Sep. 30, 2021
(86) PCT No.: PCT/EP2021/076969

§ 371 Date: Apr. 4, 2023

Related U.S. Application Data
(60) Provisional application No. 63/088,695, filed on Oct. 7, 2020.

(51) Int. Cl. C12M 1/34; C12M 1/00; G01N 35/00; G01N 35/04; C12M 1/36

(52) U.S. Cl. CPC C12M 41/36; C12M 23/22; G01N 35/00732; G01N 35/04; C12M 41/48; C12M 23/50; G01N 2035/0401

(57) ABSTRACT

A system for capturing an image of a plated culture dish. The system includes an imaging device having a camera with a telecentric lens adapted to capture an image of the plated culture dish, a minor adapted to ensure that a label on the side of the plated culture dish is captured in an image of the plated culture dish that is captured by the imaging device. The system further includes at least one light system for illuminating the plated culture dish for image capture. The mirror is placed adjacent to the side of the plated culture dish on which the label is placed and at least a portion of the minor extends beneath a bottom portion of the plated culture dish at the side of the plated culture dish.

### CROSS REFERENCE TO RELATED APPLICATION

This application claims the benefit of priority from U.S. Provisional Application No. 63/088,695 filed Oct. 7, 2019, which is incorporated by reference herein.

### BACKGROUND OF THE INVENTION

### Field of the Invention

Described herein is a system for obtaining an image of a plated culture dish using an imaging device having a telecentric lens.

### Description of the Related Art

Plated cultures are a common technique for evaluating and testing samples for evidence of microbial contamination. Various types of plated culture dishes are popular to prepare microbiological and cell cultures from such samples for research and analysis in a number of fields. Examples of the vessels for the inoculated culture media include petri dishes, microtiter or multi-well plates as well as high-density format plates, such as 384-, 864- and 1536-well plates.

The plated culture dishes typically contain media that supports microbial growth on the plated culture dish. After the plated culture dish is inoculated with sample, the plated culture dish is incubated to allow formation of colonies of any microbial contamination in the sample. Some media are selective such that only certain types or strains of microorganisms grow on the culture media in the plated culture dish.

The incubated plates are inspected to ascertain whether microbial growth has occurred. When colonies are observed, a portion of the colony of interest is picked and subjected to further analysis to learn more about the microorganisms. Manually inspecting and picking colonies of interest is time consuming and requires the use of microbiologists for this highly skilled work. Increasingly, automation is being applied to inspecting plated culture dishes to determine if there is evidence of colony formation and/or microbial growth. Such automation typically involves obtaining an electronic image of the plated culture dish and displaying such image to a microbiologist who can identify colonies of interest and control the system to pick a portion of such colony for testing. Alternatively, the image data can be evaluated and processed against a set of rules to automatically identify one or more colonies of interest.

Capturing an electronic image of the sample culture to detect microbial growth typically requires a standard 50-55 mm f1.4 photographic lens coupled to a camera. However, such systems have poor sensitivity, even when coupled to efficient cameras, so that many cultures still require imaging times of tens of minutes or more, and suffer from other issues such as vignetting (unwanted darkening) and lateral distortion effects that can cause the image to be a less than completely true image of the sample culture. However, such distortion effects in such systems did provide some ability to get image information from the side of the plate. The disadvantages have been overcome with the use of imaging systems having a telecentric lens, which provides a true top view of the culture plate. The telecentric lens is also an economical alternative to other lenses in such systems. However, in systems having the telecentric lens, the direction of the light rays incident upon the plated culture dishes is such that it lacks the distortion that provides a useful image of the sides of the plated culture dish. Therefore, to effectively deploy a telecentric lens when obtaining images of plated culture dishes, further improvements are required.

### BRIEF SUMMARY

The system and method described herein addresses the above problems by providing an intelligent imaging system having a telecentric lens that provides automatic, high-resolution digital imaging. Moreover, the imaging system described herein can be combined with an incubator to fit seamlessly into an automated lab environment or be a stand-alone unit working with a lab operator.

As noted above, when a telecentric lens is used to image an object such as a plated culture dish, the direction of the light rays incident upon the plated culture dishes is such that it cannot provide a clear image of the sides of the plated culture dishes. The side of a plated culture dish may contain useful information, such as a label that can be used as a fiducial mark that is used to align the plated culture dish in the imaging apparatus. The label can also carry barcode information identifying the plated culture dish and other information such as the culture media type, sample type, sample date, etc.

Fiducial markings are useful because the plated culture dishes are typically brought to the imaging apparatus to obtain images of the plated culture dishes several times during the incubation cycle. In order to automatically assess whether or not microbial growth has occurred in the cultured sample carried by the plated culture dish, and to what extent, the plated culture dish must be evaluated on a pixel-by-pixel basis to determine if there have been changes in the pixels from an earlier image to a later image that are indicative of microbial growth. In order to make a successful pixel-by-pixel comparison, the pixels in the earlier image must be aligned with the pixels in the later image.

The need for pixel alignment in automated systems and methods for evaluating plated culture dishes for indications of microbial growth is known. For example, in the imaging apparatus described herein, the colonies on the plate are imaged according to the methods described in: 1) PCT/US2016/028913 Apr. 22, 2016 entitled “Colony Contrast Gathering,” which published as WO/2016172527; and 2) PCT/EP2015/052017 entitled “A System and Method for Image Acquisition Using Supervised High Quality Imaging” which was filed on Jan. 30, 2015 and was published as WO2015/114121, and which applications are incorporated herein by reference. As described in these references, the plated culture dish that is inoculated with sample is incubated. After a time, an image of the inoculated culture dish is obtained. The plated culture dish is then returned to the incubator for additional incubation. After another period of time the plated culture dish is retrieved and imaged again. The earlier image is then compared with the later image on a pixel-by-pixel basis. As noted above, to do this, the imaging apparatus must align the pixels in the first image with the pixels in the second image to identify changes in the pixels that might be indicative of microbial growth.

The contrast of the different colonies against the culture medium provides the ability to discriminate colonies to facilitate automated colony pick. In this regard, the bar code fiducial information can be used not only to align pixels in an image of a plated culture dish at time tₓ with the pixels in a later image (an image obtained at time tₓ₊₁, but the fiducial information provided by the label can be referenced to determine the location of colonies of interest in an apparatus that is used to pick the colonies of interest for downstream tests such as microbial identification and antibiotic susceptibility.

As described above, after the initial image of the plated culture dish is obtained, the plated culture dish is incubated for a period of time to allow microorganisms on the plate, if present, to grow. In a further example of the system described herein, the system performs the automated steps of: i) positioning the plated culture dish on a stage for a culture dish; ii) obtaining an image of the plated culture dish positioned in the stage; iii) obtaining the identification of the culture dish; iv) comparing the image obtained by the imaging device with the stored initial image of the plated culture dish for obtaining information regarding the location of the selected colony of microorganisms (to inform the pick tool device on the location of the colony to be picked); and, optionally, vi) obtaining the processing instructions regarding the processes to be performed on the selected colony of microorganisms. By comparing the image of the culture dish when it is placed in the pick tool device with the initial image, the location of the selected colonies can be obtained automatically, for example by computerized image comparison.

The label, or more particularly the sides of the label, are used as a reference to locate the plated culture dish in the imaging apparatus to facilitate the pixel-by-pixel alignment of an image of the plated culture dish obtained at a first, earlier time with an image of the plated culture dish at a second, later time. As noted above, if the label is used to facilitate this alignment, the imaging device must be able to locate the label in the image information.

The label sides alone are insufficient to both align the pixels in the images obtained at different times and to identify the coordinates of colonies of interest over time. Using a machine vision apparatus, another reference point such as the center of the dish is detected from which dish coordinates can be determined. The location of colonies on the dish can be determined in reference to their relative distance from the center and angular offset to the label zero offset. Once the relative location of a colony of interest is determined, then the plated culture dish can be moved to another system where the following two steps are performed. The dish is centered, for example, by mechanical means. The barcode zero offset is detected, for example by rotating the dish while having a fixed sensor to detect the presence of the barcode label and scan the barcode with a barcode scanner. At this point the center of the dish is known and the barcode zero offset is known and therefore the location of the previously referenced colonies can easily be calculated as they are stored as distance to the dish center and angular offset to the barcode label. The automated system as it is described herein does not need a camera or computer vision system in the second system (colony picking system in this example), or any other system where the colony position information is required. The angular offset used in this example is with reference to the barcode label but it could reference any unique fiducial feature of the dish or applied to the dish as noted above.

To use the label for pixel alignment, at least one of the lateral ends of the label must be clearly captured by the imaging apparatus using the telecentric lens. Because the label length and dish curvature are known, the system can calculate the location of the other label end, and in turn calculate the label center. The coordinates of any object on the plate can be determined with knowledge of the plate center and the label center. As noted above, there is a need in the art for improved imaging systems that deploy a telecentric lens that provides monitoring capabilities for plated culture dishes, especially with little to no operator intervention. In order to deploy a telecentric lens in a system where a label on the side of a culture dish is used as an alignment fiducial, the use of the mirror is critical to allow the telecentric lens to obtain an image of the label.

In one aspect, the system described herein provides a system for capturing an image of a plated culture dish. The system has: i) an imaging device having a camera with a telecentric lens adapted to capture an image of the plated culture dish; ii) a mirror adapted to ensure that a label on the side of the plated culture dish is clearly visible in the image captured by the imaging device; and iii) at least one light system for illuminating the plated culture dish for image capture. Optionally, the mirror is placed relative to the plated culture dish on which the label is placed such that, vertically, the mirror is below the bottom of the plated culture dish. However, laterally, at least a portion of the mirror extends beneath a bottom portion of the plated culture dish (i.e. a portion extends into the perimeter defined by the plated culture dish that sits above the mirror). It follows then that at least a portion of the mirror extends laterally beyond the perimeter of the plated culture dish that sits above the mirror.

Optionally, the image capture system described herein may have a telecentric lens module that aligns and fixes the position of the telecentric lens and the camera of the imaging device with respect to the plated culture dish. The telecentric lens module comprises one or more brackets and one or more plates.

Optionally, the image capture system described herein may be part of an integrated incubator and image capture module that regulates the incubator atmosphere and obtains high-resolution digital images of sample specimens. Optionally, the image capture module is equipped with a stage that receives the plated culture dishes conveyed from the image capture module. The stage is provided with a scanner that will scan the label on the side of the plated culture dish. The stage is also provided with plate bumpers, one of which is hinged and moves from an open position when the plated culture dish is received in the stage to a closed position when the scanner determines that the label is within a predetermined orientation relative to the stage. The purpose of the stage is to ensure that the orientation of a label on a plated culture dish received into the incubator is somewhat consistent from plate to plate. By maintaining the labels on the plated culture dishes within a predetermined range of acceptable orientations, it is easier to position the plated culture dish in the imaging apparatus such that the label aligns with the mirror. This also provides for better uniformity of imaging conditions within the plate. Specifically, the imaging apparatus does not provide completely uniform illumination of the plate surface. By placing the plate in the same position relative to the imaging apparatus every time an image is obtained, each region or area of the plate surface is subjected to identical imaging conditions over time (i.e., for region “x” the imaging conditions “y” are identical for the image at time tₓ, tₓ₊₁, tₓ₊₂, etc.)

If the plated culture dishes are received into the imaging apparatus without some predetermined orientation, then the orientation is essentially random and the imaging apparatus will have to spend time and processing resources to place the plated culture dish in an orientation where the label will align with the mirror. Since the position of the label can be anywhere on the plate circumference in this scenario, a plated culture dish might need to be rotated 180 degrees or more so that the label will align with the mirror. If the plated culture dish is delivered into the imaging apparatus with the label orientation within a predetermined range relative to the sensor that will read the label when the plated culture dish is received into the imaging apparatus, then the imaging apparatus will expend less time reorienting the plated culture dishes with the label thereon prior to imaging.

Further advantages will be realized by various aspects of the system and method described herein and will be apparent from the following detailed description. One of the advantages of the system described herein is the integration with automated platforms for plate inoculation, providing end-to-end automation for inoculation of sample onto plated media, streaking of sample on to media and incubation of inoculated media for growth of target microorganisms. The present system is flexible and can also handle plated media that have been inoculated manually.

### BRIEF DESCRIPTION OF THE DRAWINGS

The system and method described herein will be better understood from the Detailed Description and from the appended drawings, which are meant to illustrate and not to limit what is described.

FIG. 1 is an image obtained using a camera with a non-telecentric lens of a plated culture dish with a barcode label on the side of the plated culture dish;

FIG. 2 is an image obtained using a camera with a telecentric lens of a plated culture dish with a barcode label on the side of the plated culture dish;

FIG. 3 is an exemplary schematic of the reflection of a light beam on a convex mirror used in the image capture system described herein;

FIG. 4 is an exemplary schematic showing the reflection of light beams on a convex mirror of the system described herein, where the mirror is placed on the adjacent side and under plated culture dishes of different sizes;

FIG. 5 is an exemplary image of a plated culture dish with a barcode label along the side of the plated culture dish, the image obtained using the system described herein having a camera with a telecentric lens and an arced mirror placed adjacent to the side of the plated culture dish on which the label is placed and at least a portion of the mirror extends beneath a bottom portion of the plated culture dish;

FIG. 6 is a perspective view of an interior portion of an image capture module of the system describe herein that is integratable with an incubator;

FIG. 7 is a back perspective view of the image capture module of the system described herein;

FIG. 8 is a detail view of the plated culture dish ingress into the image capture module of FIG. 7;

FIG. 9 is a side perspective view of the image capture module of the system described herein;

FIG. 10 is a detail view of a buffer position of the image capture module illustrated in FIG. 9;

FIG. 11 is a detail view of a scanning station of the image capture module illustrated in FIG. 9;

FIG. 12 is a detail view of an indexing station of the image capture module in FIG. 9;

FIG. 13 is a detail view of a lid manipulator of the image capture module in FIG. 9;

FIG. 14 illustrates the plated culture dish being advanced into the indexing station;

FIG. 15 illustrates the glass plate over which the plated culture disc is placed by an indexing disc;

FIG. 16 illustrates the indexing disc;

FIG. 17 illustrates an indexing disc mechanism according to an aspect of the system described herein;

FIG. 18 is a cross-sectional view of the image capture system of an aspect of the system illustrated in FIG. 6, where the cross-section 18-18 bisects the telecentric lens;

FIG. 19 is a detail view of the imaging chamber of the image capture system illustrated in FIG. 18;

FIG. 20 is a magnified view of a portion of FIG. 19 illustrating the indexing station in the imaging station;

FIG. 21 is a top down view of the image capture system showing the plated culture dish and the mirror at least partially underneath the plated culture dish;

FIG. 22 illustrates the imaging disk exit position of the imaging apparatus of FIG. 6;

FIG. 23 illustrates the plated culture dish exiting the imaging apparatus described herein according to one aspect;

FIG. 24 illustrates a flow chart for the method described herein, both for locating the plated culture dish on the glass dish support and locating the label center relative to the dish center to assign coordinates to object in the image of the culture dish;

FIGS. 25A and 25B illustrate a polar image created from the image of a label;

FIG. 26A illustrates the angular location of the label relative to the plated culture dish center that is used to determine the coordinate system of the plated culture dish;

FIG. 26B projects the geometric analysis in FIG. 26A on an image of a plated culture dish on a glass plate.

FIG. 27 illustrates masked areas representing the plated culture dish and the glass dish support for the plated culture dish;

FIG. 28 illustrates a polar image of the plated culture dish edge; and

FIG. 29A and 29B illustrate a plated culture dish lift and a plated culture dish scan lift, respectively.

### DETAILED DESCRIPTION

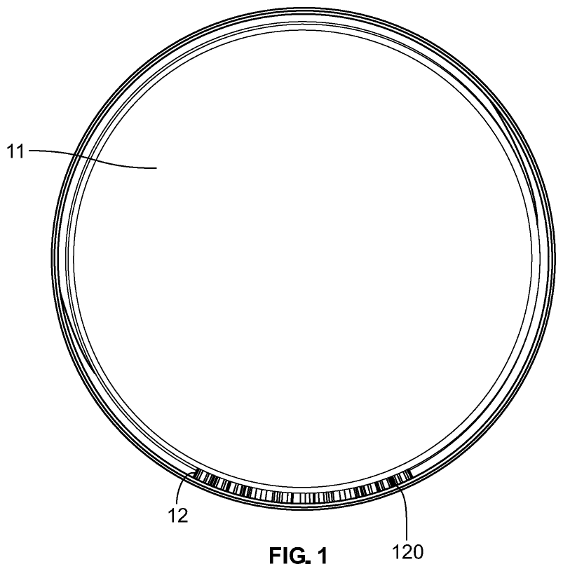

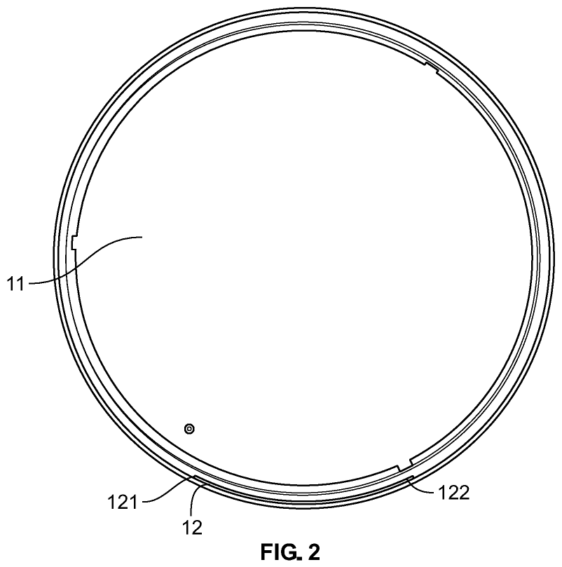

FIG. 1 is an image of a plated culture dish 11 having a label 12 on the outside of the plated culture dish. Optionally, the label 12 has a barcode 120 thereon. No microbial growth is evident in the image. The image was obtained using a camera having a non-telecentric lens. One non-limiting example of such a lens includes, for instance, a standard 50-55 mm f1.4 photographic lens. The label 12 with the barcode 120 is clearly visible on the image. However, with reference to FIG. 2, when a camera with a telecentric lens is used to obtain the image of the same plated culture dish 11, the label 12 with the barcode 120 is not very visible in the image.

As noted above, the label is used as a fiducial to facilitate pixel alignment between images of the plated culture dish taken at different times. The label optionally has barcode information. The barcode can contain information identifying the plated culture dish, the type of culture media, the sample, etc. The ends 121, 122 of the label must be clearly visible on the image to effect pixel alignment between images obtained at different times.

In order for the imaging apparatus to obtain the information about the label that will facilitate alignment, a mirror is positioned relative to the plated culture dish to reflect the label on the side of the plated culture dish. In some aspects, the mirror is placed below the bottom of the plated culture dish. At least a portion of the mirror extends laterally under the plated culture dish such that that portion of the mirror is within a perimeter defined by the plated culture dish held above the mirror. A portion of the mirror extends beyond the perimeter defined by the plated culture dish held above the mirror. Optionally, the mirror is a convex mirror.

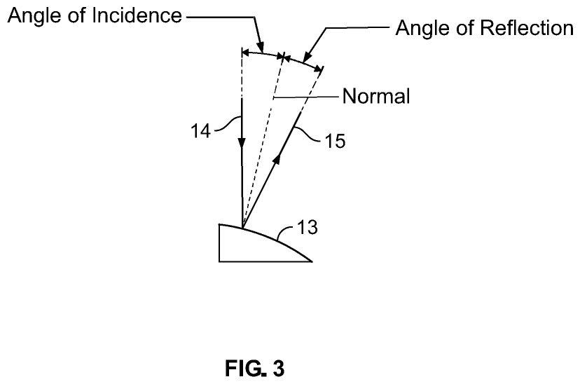

FIG. 3 shows an exemplary schematic of the reflection of a light beam on a convex mirror 13, such as one that is described herein. Light rays 14 are directed essentially vertically 14 downward onto the convex mirror 13. When the light rays 14 impinge on the spherical surface of the mirror 13, they are reflected 15 according to the well-known principle that, for a reflective surface, the angle of incidence is equal to the angle of reflection.

As noted above, for image alignment, at least one of the edges of the label is detected. This edge detection is used to place the label center relative to the center of the plated culture dish. The label center is determined based on some a priori knowledge (i.e., label length, mirror curvature and dish curvature). This information is then used to understand the relative placement of objects in the plated culture dish. The next time the plated culture dish is brought into the imaging apparatus, at least one of the edges of the label is again determined. Based on the information regarding the label center relative to the dish center, the software can calculate the offset between the earlier image and the later image. Using that offset, the imaging apparatus aligns the pixels in the first image with the pixels in the second image.

Because the center of the label is used for alignment and the label center is determined by detecting the position of the label edges (or at least one label edge) on the mirror, a high-quality image of the label edges is required. Because reflections from a highly polished mirror surface may distort or blur the image of the label edges, a less than highly polished mirror surface mitigates some of the distortion and blur. However, if an image of label information, e.g., barcode information or other information carried by the label is sought, a highly polished mirror having a polished specular surface that provides specular reflection may be preferred. Based upon the label information sought, one skilled in the art can select the desired type of reflection (i.e., specular or diffuse).

The plated culture dishes can come in different sizes and the labels of interest may be placed in different positions on the plated culture dishes. A person of ordinary skill in the art is able to determine the dimensions of the mirror, the bend of the mirror, and the placement of the mirror adjacent to and under each of these plated culture dishes that would be acceptable to provide a reflection of the label for a given size of a plated culture dish and the placement and size of the label of interest on the plated culture dish.

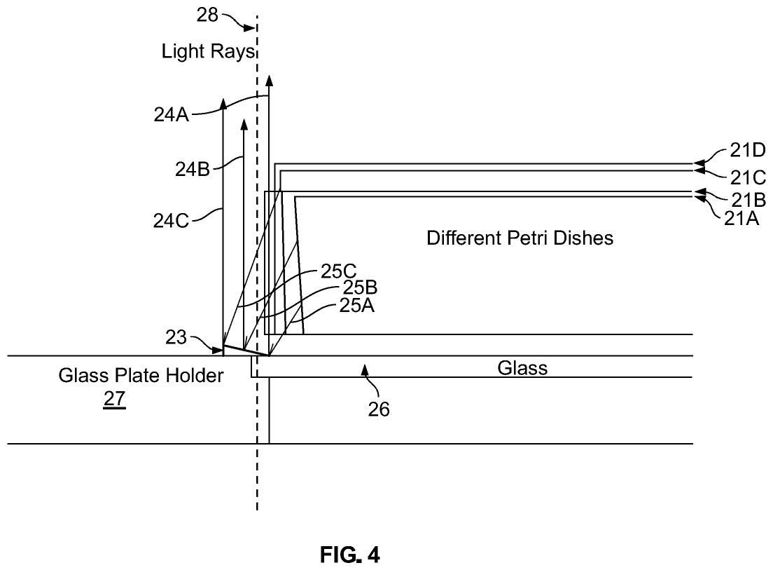

FIG. 4 illustrates an exemplary schematic showing the reflection of light beams on a convex mirror 23, where the mirror is positioned under the plated culture dishes of different sizes 21A-D (only a portion of each plated culture dish is illustrated in FIG. 4). As illustrated in FIG. 4, a portion of the mirror 23 extends laterally into a perimeter 28 defined by the plated culture dish 21A-D overlying the mirror 23. Another portion of the mirror 23 lies laterally outside this perimeter. As seen from the schematic, the placement and size of the mirror 23 is configured to cooperate with plated culture dishes of different diameters and different heights to provide a reflection of the label on the plated culture dish (the different dish configurations are 21A-D). The mirror 23 is placed above a transparent (e.g., glass, plexiglass) culture dish window 26 that is held in place by a plate holder 27. Although the culture dish window 26 is described herein as glass, one skilled in the art will appreciate that other transparent materials (e.g., acrylic glass, plexiglass, etc.) might also be used, provided that such materials are sufficiently transparent and non-reflective. The plated culture dish 21A-D is held above the glass plate 26 by the indexing disc (described in detail later herein). The glass plate 26 allows the plated culture dish 21 A-D to be illuminated from below the plated culture dish 21 A-D. As illustrated, the glass plate 26 extends beyond the lateral limits of the plated culture dish 21A-D. The glass plate is supported by the plate holder 27, which is opaque and surrounds the perimeter of the glass plate 26. Depending on the dimensions of the plated culture dish, such differences being illustrated by as different dish profiles 21A, 21B, 21C and 21D, the position of the mirror 23 can either be configured to reflect the image of a label on the side of plated culture dishes with different diameters, or optionally be adjusted to reflect the image of such label on plated culture dishes 21A-D having different diameters. Either way, the reflection of the side of the plated culture dish is captured by the telecentric lens which receives light essentially vertically from the object being imaged. Specifically, plated culture dish 21A has a first height and a first diameter, plated culture dish 21B has a larger diameter than 21A but has about the same height and plated culture dishes 21C and 21D have still larger heights but diameters greater than the diameter of 21A but less than the diameter of 21B. The image of the side of the plated culture dishes 21A, 21B, and 21C that are reflected by the mirror 23 are represented by 25A, 25B and 25C, respectively. The respective label reflections, represented by 24A, 24B, and 24C are directed toward and received by the telecentric lens, allowing the telecentric lens to capture the image of a label (not shown) on the side of the plated culture dishes 21A, 21B, and 21C. That label information is used as described above to effect pixel-by-pixel image alignment of the plated culture dish between two images of the plated culture dish taken at different times with an intervening incubation step.

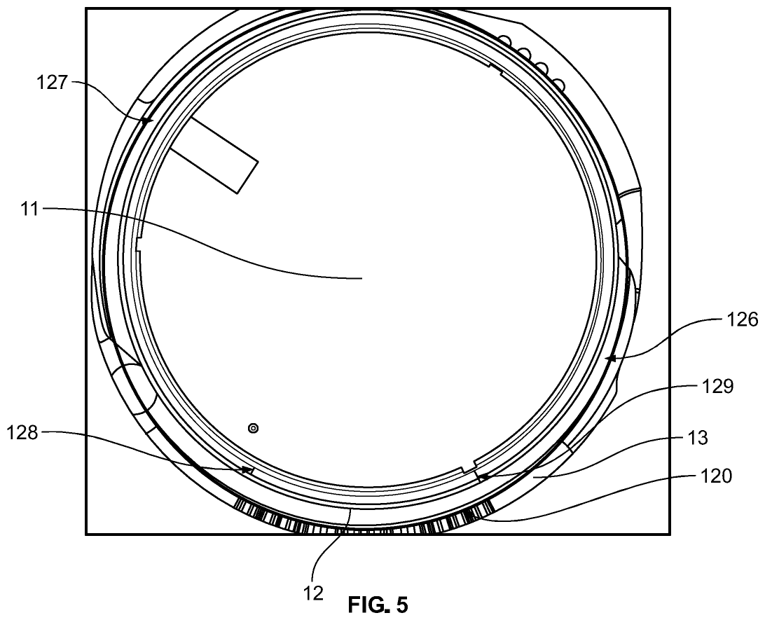

FIG. 5 is an exemplary image of a plated culture dish 11 with a label 12 along the side of the plated culture dish 11. The label 12 has a barcode 120 thereon. However, as noted above, if the label 12 is used for image alignment, there is no requirement that a barcode 120 be on the label 12. The label 12 can be placed either on the inside surface of the plated culture dish 11 or the outside surface of the plated culture dish 11.

The image is obtained, according to one aspect described herein, using a system having a camera with a telecentric lens and an arc-shaped mirror 13 placed underneath the plated culture dish and to the side thereof. The image apparatus orients the plated culture dish 11 such that the label 12 aligns with the mirror 13. As seen from the image in FIG. 5, the label 12 with the barcode 120 is clearly visible, as is its reflection in the mirror 13. This ensures that an image of the label 12 can be captured by the imaging apparatus with the telecentric lens and used for pixel-by-pixel alignment of a first image of the plated culture dish 11 with a second image of the plated culture dish taken at a later point in time with an intervening incubation step.

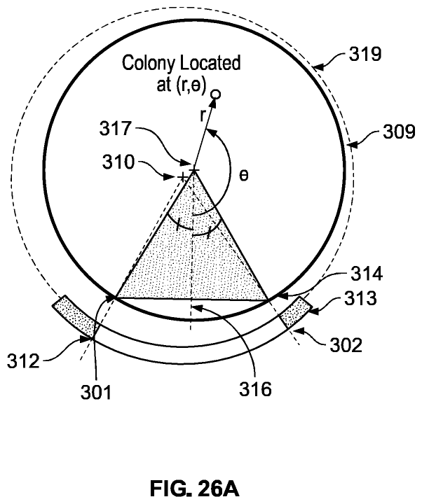

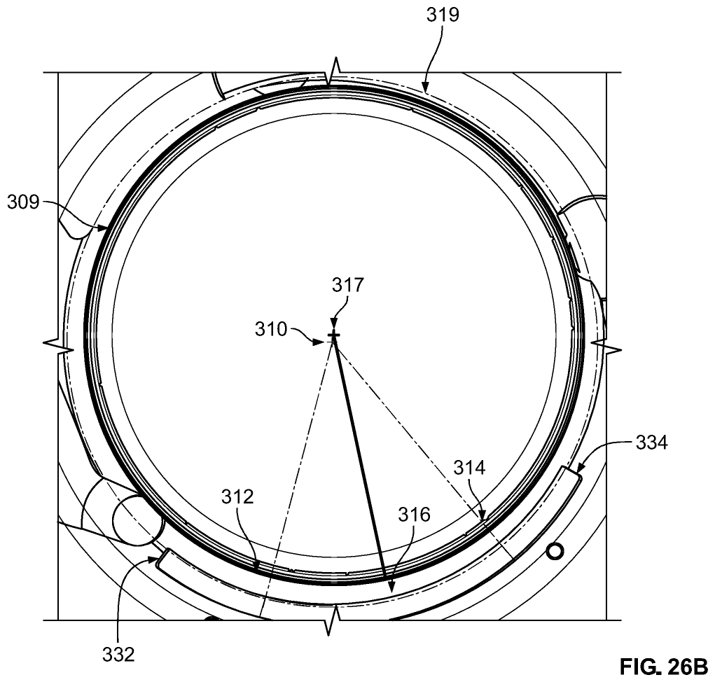

The plated culture dish is placed above a glass plate 126. The coordinate space of an acquired image of the plated culture dish 11 is determined by label detection. Specifically, the precise location of both lateral ends 128, 129 (also 312, 314 in FIG. 26A) of the label 12 along the dish contour is determined. From this the label center is determined. Those locations are captured as angular coordinates using the center of the culture dish 11 as the origin. These label angular coordinates and the dish center shall allow to precisely locate in the plate referential the colonies marked on the image and to be picked later by IdentifA or any other manual or automatic system. IdentifA is a system provided by BD Kiestra™ lab automation solutions (Becton Dickinson and Company) (BD). The dish contour and dish center are unknown prior to dish detection.

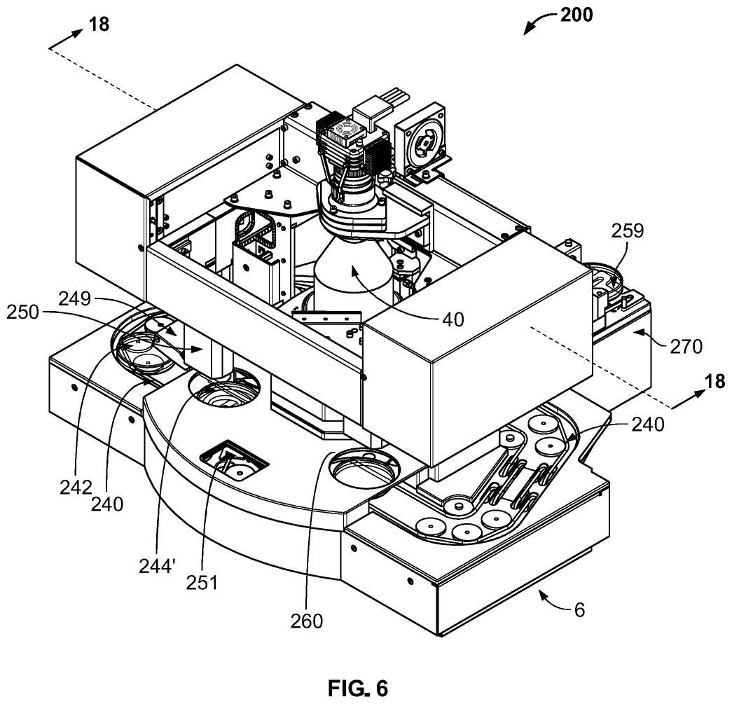

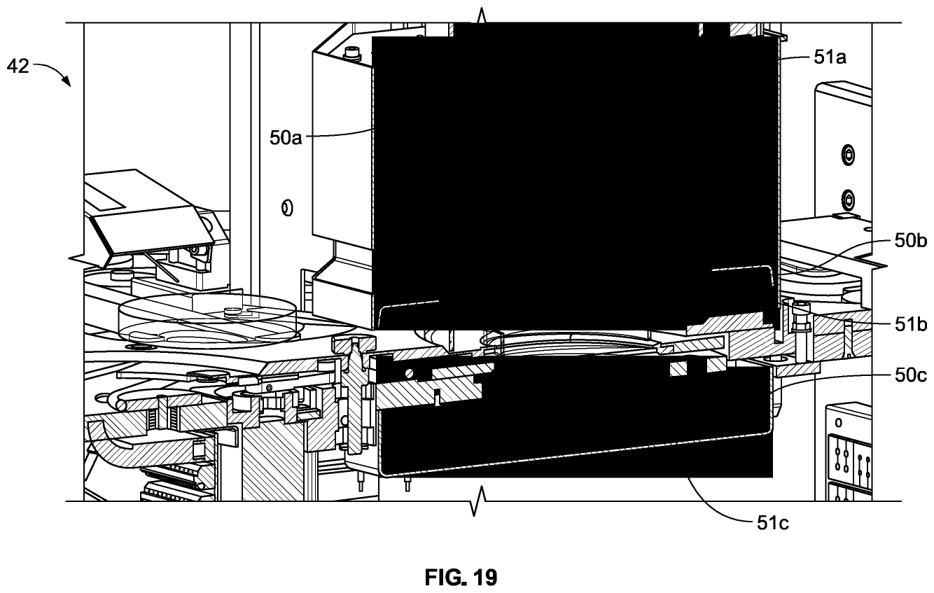

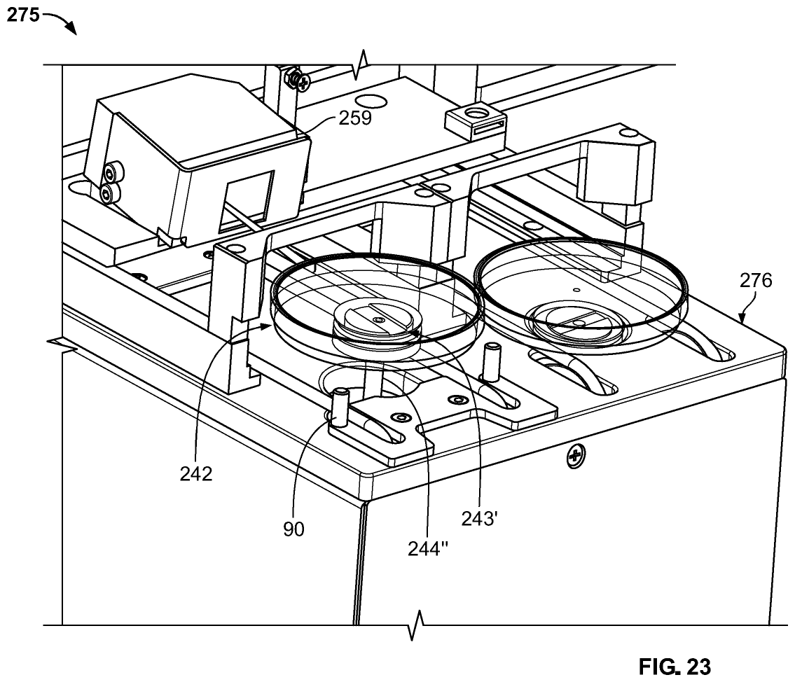

FIG. 6 is a perspective view of an interior portion of an aspect of the image capture module 200 described herein that is integrated with an incubator. In particular, FIG. 6 illustrates the conveyor 240 from the incubating system, through the imaging unit and back to the incubator. As illustrated in FIG. 6, the plated culture dish 242 travels along conveyor system 240. When the plated culture dish 242 reaches a specified location, the lid manipulator 250 will remove the lid from the plated culture dish 242. The label is then read by a reader (i.e., bar code scanner or RFID reader) 249, while the plate is rotated by a scan lift. The plated culture dish 242 is then moved onto an indexing disc 251. The plated culture dish 242 is advanced, via rotation of the indexing disc 251, into an imaging station 253 (FIG. 19). The indexing disc 251 moves the plated culture dish into position under image capture unit, which is described in detail in U.S. Application Publication No. 2015/0299639 A1. After imaging, the plated culture dish is rotated to position 260, where the lid is placed back on the culture dish. The plated culture dish 242 is then off-loaded back onto the conveyor 240 where it is conveyed back to unloading station 270. Unloading station 270 is located in the incubator cabinet (not shown) to which the image capture module 200 is mounted. Unloading station 270 has a scanner 259 and an indexing disc (not shown). The scanner 259 determines the location of the label on the plated culture dish and another scan lift 244′ (FIG. 23) rotates the plated culture dish 242 so that the label is within a predetermined orientation relative to the robot (not shown) that will off-load the plated culture dish from the unloading station.

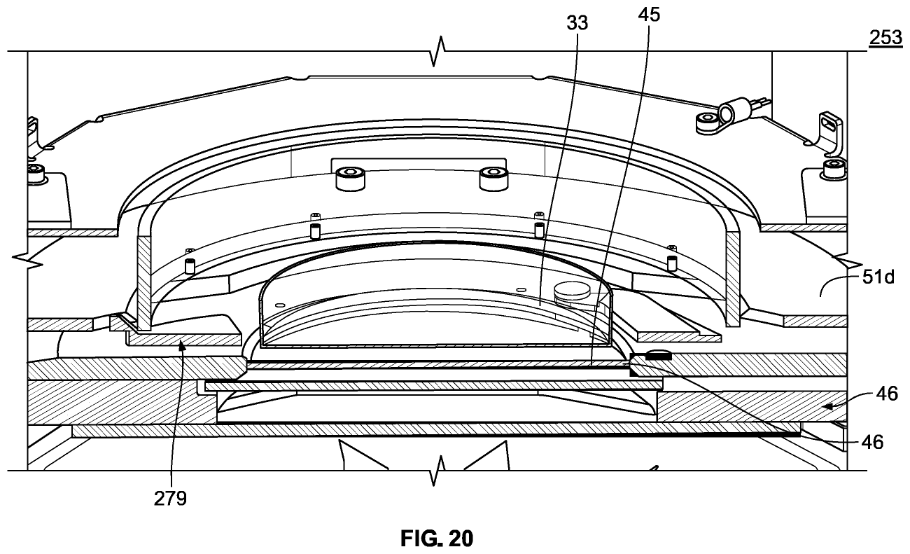

With the labels in roughly the same orientation on the plated culture dishes 242 relative to the unloading and loading robot, the degree to which the plated culture dishes 242 will potentially need to be rotated by the scan lift 244′ when placed in the indexing disc 251 to ensure that the labels will align with the mirror 33 in the imaging station 253 (FIG. 20) is reduced. Once the sensor determines the location of the label, software controls the rotation of the plated culture dish to place the label in the desired orientation relative to the robot. The efficiency provided by this approach is described above.

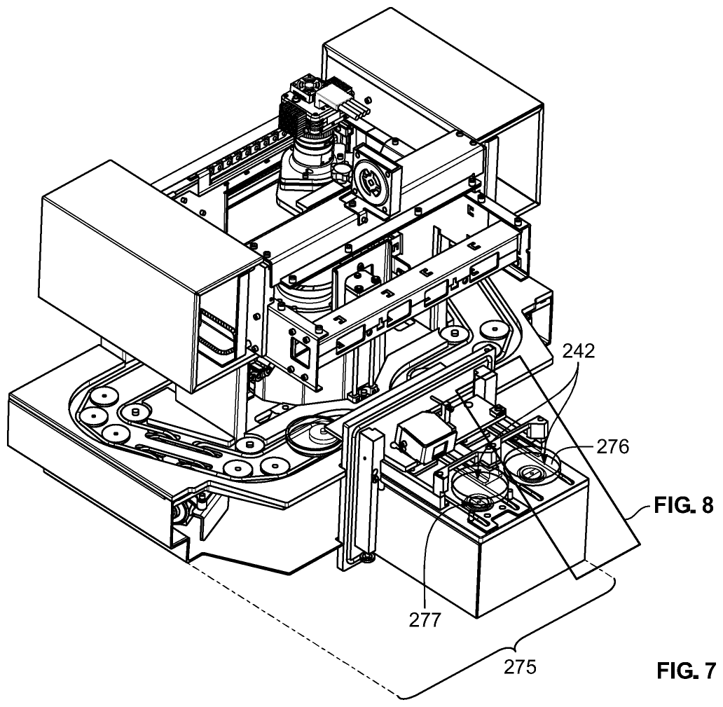

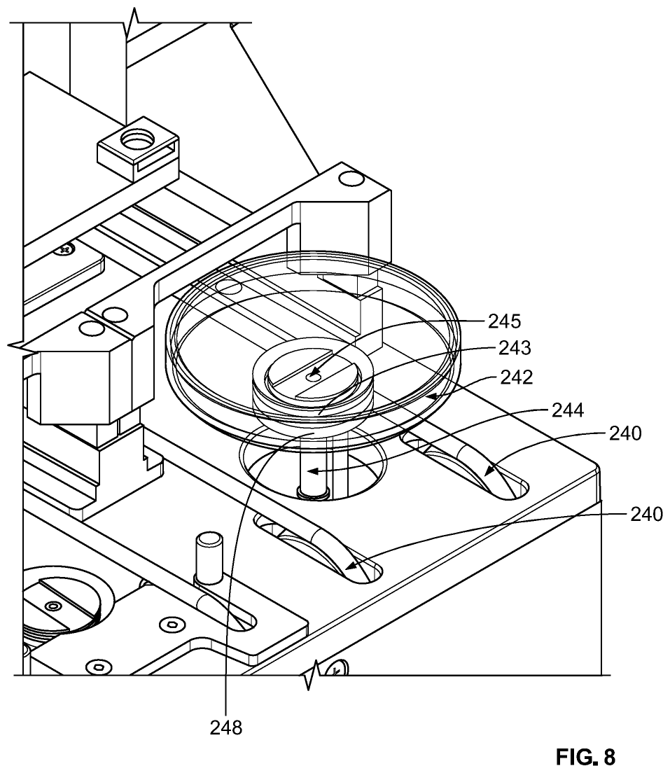

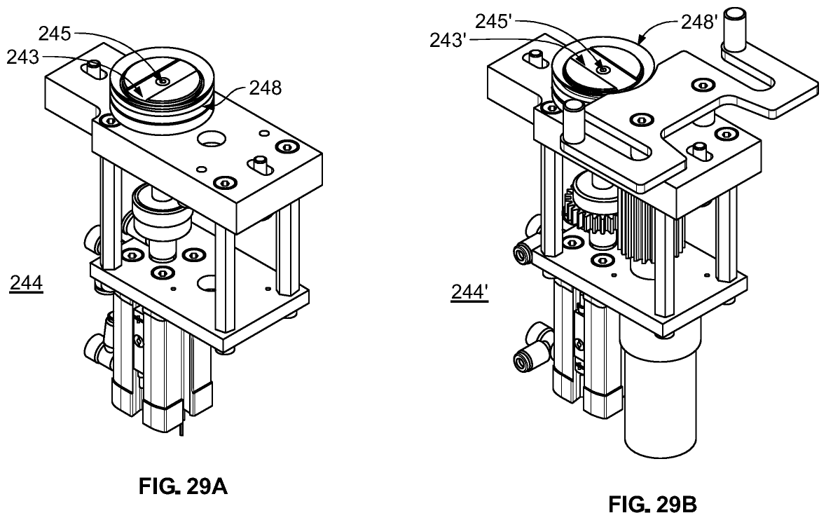

FIG. 7 is a rear perspective view of the image capture module illustrated in FIG. 6. FIG. 7 illustrates the entrance subsystem 275 where the plated culture dishes 242 ingress 276 into and egress 277 from the image capture module. The apparatus for culture dish ingress 276 is illustrated in detail in FIG. 8. The apparatus confirms that a plated culture dish 242 has been delivered onto the suction cup 243 of the culture dish lift 244. The suction cup 243 is supported by a stationary platform 248. The placement of the plated culture dish 242 on the culture dish lift 244 is confirmed by sensor 245. The lift 244 as described herein is to be distinguished from scan lifts 244′/244″ in that the lift 244 moves the plated culture dish up and down whereas the scan lifts 244′/244″ move the plated culture plate 242 up and down and also rotate the plated culture dish. Lift 244 is illustrated in FIG. 29A and scan lift 244′/244″ is illustrated in FIG. 29B. The lift in FIG. 29A has a non-rotating platform 248 and therefore no rotating mechanism, just a lift mechanism. The scanning lift mechanism in FIG. 29B is illustrated with a rotating platform 248″ controlled by the rotating and lift mechanism of scanning lift 248′.

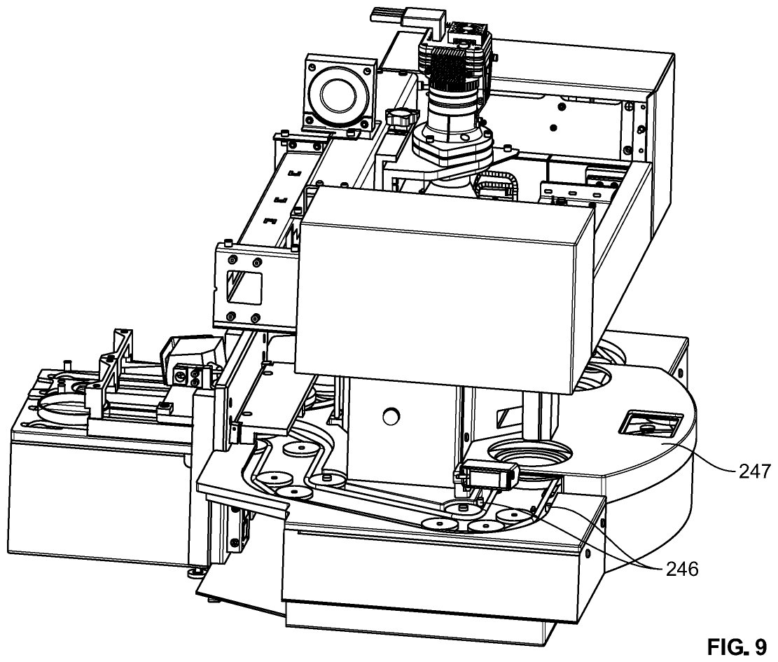

FIG. 9 is a side perspective view of the image capture module illustrated in FIG. 6. FIG. 9 illustrates the buffer position 246, where the culture dish (not illustrated so that the buffer position can be viewed) is held prior to indexing. The buffer position 246 holds the plated culture dish until the image capture module is ready to receive the next culture dish for imaging. FIG. 9 also illustrates the cover 247 placed over the scan lift 244′ and the indexing disc 251.

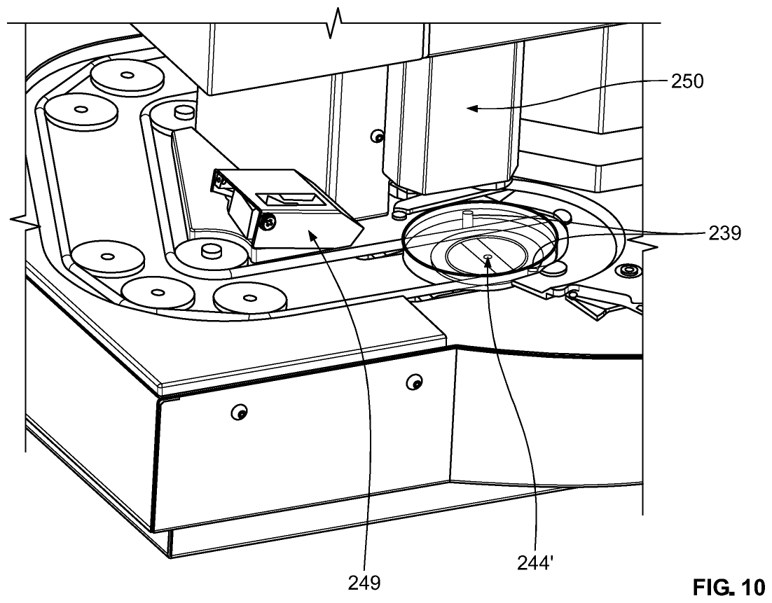

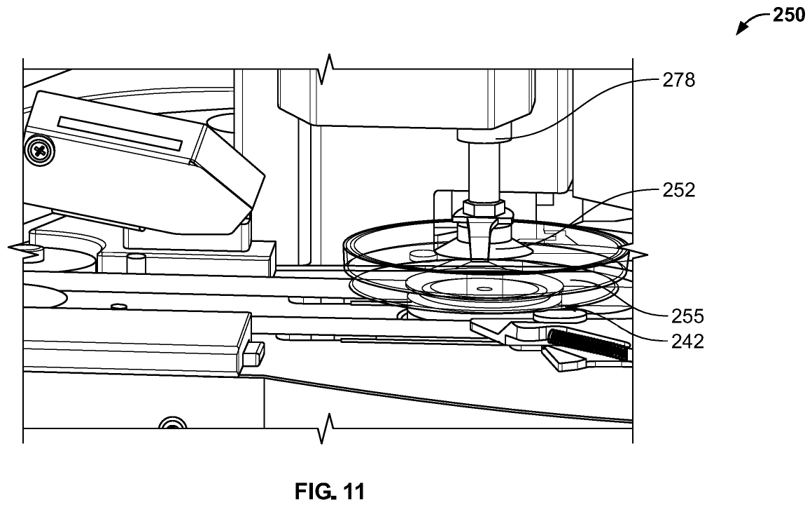

FIG. 10 is a detail view of the scanning station 239 having the scanner 249, where the culture dish is moved after it is released from the buffer position 246 in FIG. 9. The cover 247 in FIG. 9 is removed in FIG. 10. At this position there is also the culture dish scan lift 244′. The culture dish scan lift has a suction cup 243′ and a sensor 245. The suction cup 243′ is on a rotating platform 248′. The scanning station 239 is also where the lid manipulator 250 is located. The plated culture dish 242 illustrated in FIG. 10 has the lid 255 removed therefrom. FIG. 11 illustrates the lid manipulator 250 which has an arm 278 with a suction cup 252 thereon that will lift the culture dish lid 255 from the plated culture dish 242.

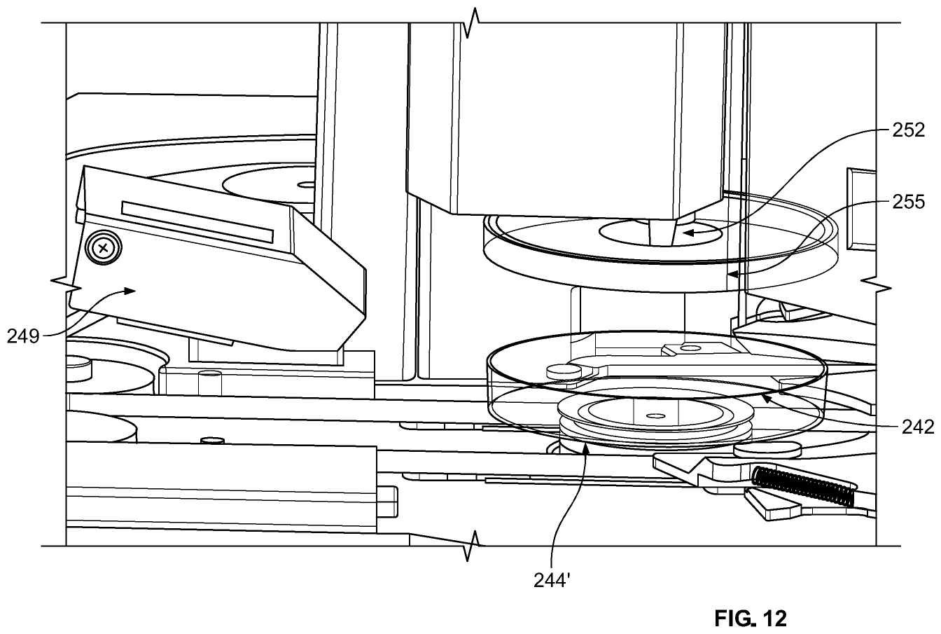

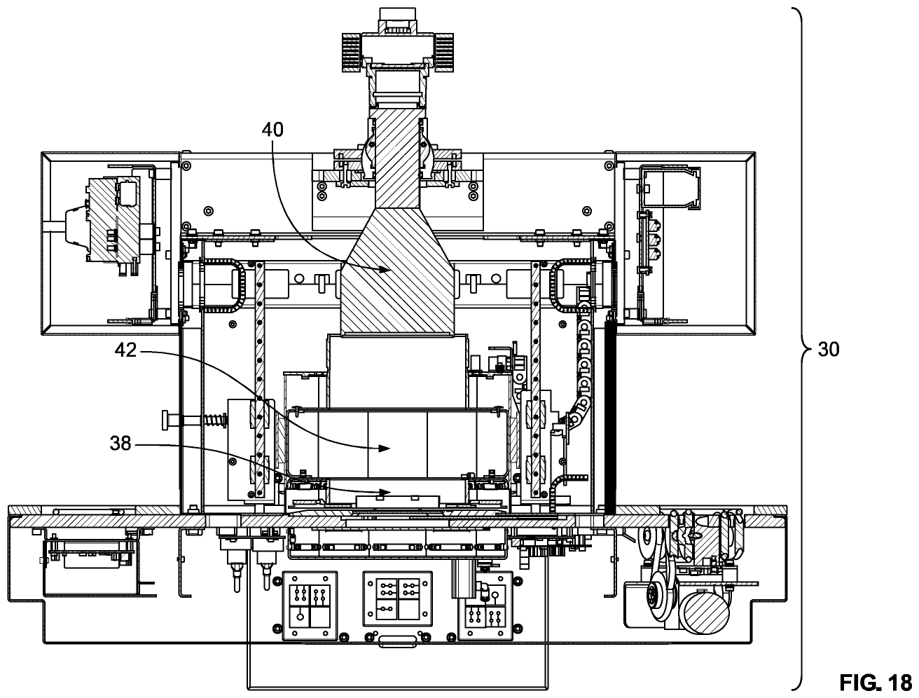

Referring to FIG. 12, the culture dish lid 255 has been lifted from the plated culture dish 242. The scan lift 244′ rotates the plated culture dish so that the scanner 249 can read the label on the side of the plated culture dish 242. Since the culture dish has been pre-oriented when it is introduced into the image capture system 30 (FIG. 18), the plated culture dish 242 is rotated only about 90 degrees to be within the field of vision of the scanner 249. When the bar code is read, the scanner sends a signal to a system controller that starts an offset timer. During the timer duration, the barcode is will be placed in alignment with a mirror in the imaging position (described below). When the timer has timed out, the culture dish lift lowers the culture dish back on to the conveyor 240 and the plated culture dish 242 is allowed to be advanced to the next position.

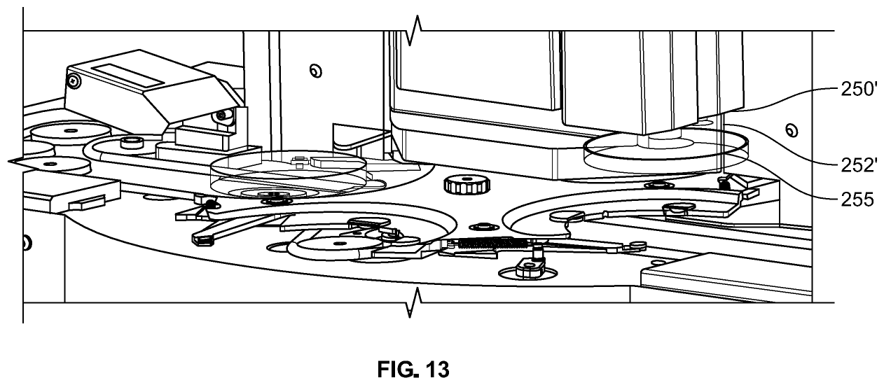

The culture dish lid 255 remains off the plated culture dish 242 while the image of the plate is obtained. Referring to FIG. 13, the lid manipulator 250 moves the lid to a second lid manipulator 250′ with a suction cup 252′ which accepts the culture dish lid 255 for placement back on to the plated culture dish 242 when the culture dish is moved into placement to receive the culture dish lid 255 after an image of the culture dish has been obtained. Lid manipulator 250′, in one aspect, is a cylinder that has three different vertical positions. The cylinder has the suction cup 252′ attached thereto. The cylinder starts in its upper position. When the culture dish lid 255 is advanced into position by the lid manipulator 250, the cylinder lowers to the second position where the suction cup 252′ contacts the culture dish lid 255. The cylinder lid then advances to the third lower position, where it is released back on to the plated culture dish 242.

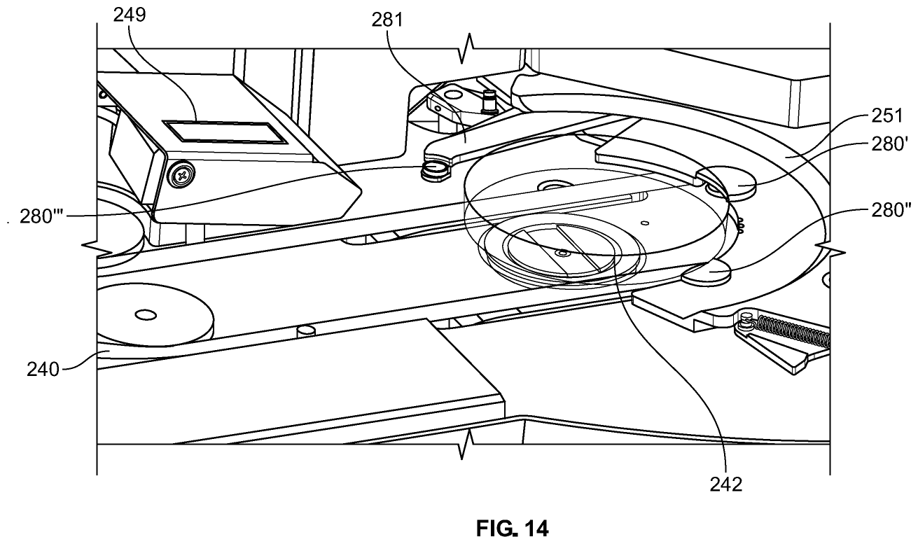

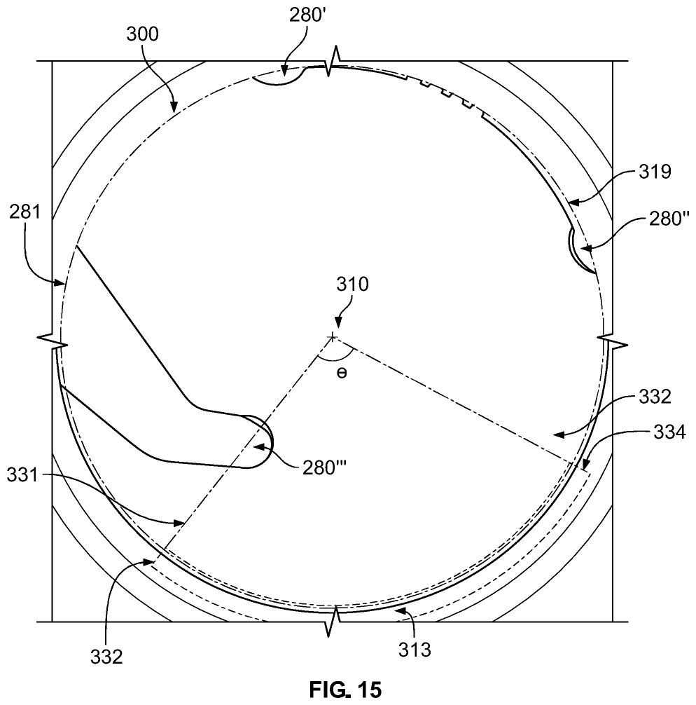

Referring to FIG. 14, after the plated culture dish 242 has been scanned by scanner 249 it is advanced by the conveyor 240 to the indexing disc 251. The indexing disc fixes the location of the plated culture dish 242 in the x-y-z coordinate space. This way the plate has a similar position and orientation each time the plate is imaged. The plate is inclined by three bumpers 280′ 280″ and 280″′. Bumper 280′ is completely fixed, bumper 280″ is fixed on a bearing (not shown) that will allow the plate to settle between the three bumpers for maximum grip. A smaller bumper 280′ is attached to a hinged arm (flipper) 281 that closes in on the culture plate 242 to fix it in place. This structure is also described with reference to FIG. 15.

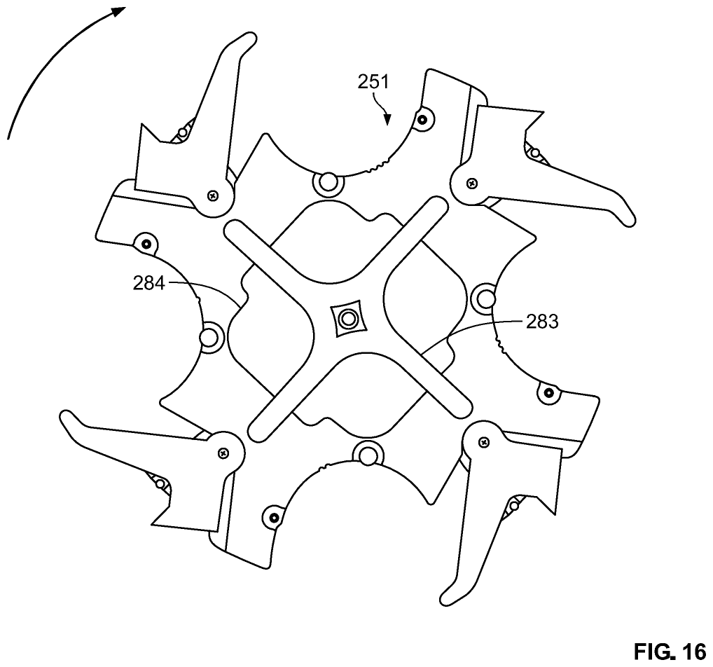

FIG. 16 illustrates the entire indexing disc 251. When the plate is aligned and an imaging position is ready to receive a plated culture dish, the indexing disc 251 will rotate 90° to move the plate to the imaging position. The indexing disc, in one aspect, has an internal mechanism for providing intermittent rotary motion (e.g., a Geneva mechanism). The mechanism illustrated in FIG, 16 has two tracks. One track 283 is for advancing the indexing disc 251. A second track 284 is used to lock the mechanism after it has advanced a plated culture dish by 90°.

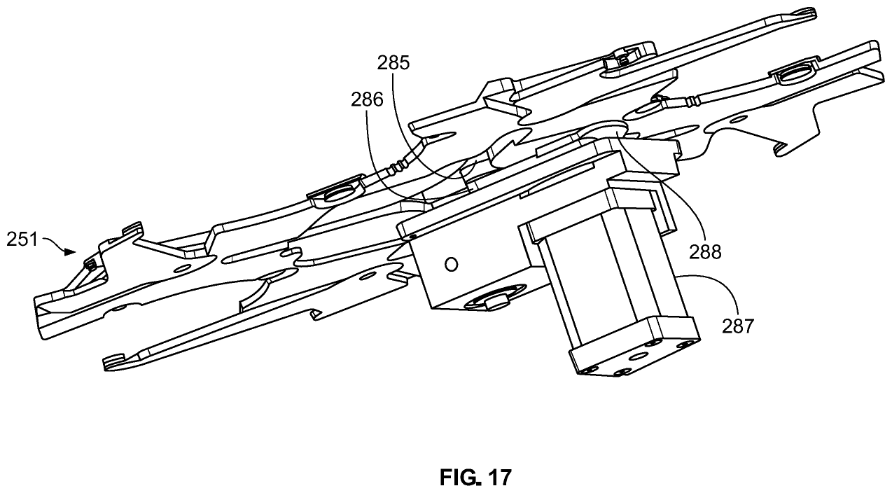

With reference to FIG. 17, the indexing disc 251 illustrated in FIG. 16 is driven by a bearing 285 in an arm 286 fixed to a stepper motor 287. The indexing disc 251 has four indexing positions and those positions are fixed through the lock 288. In one aspect, the ratio of the stepper rotation to the rotation of the indexing station is 4 to 1 (i.e., for each full rotation of the stepper motor the indexing station is advanced 90°.

FIG. 18 is a cross-sectional view of the image capture system 30 of an aspect of the system described herein, wherein the cross-section bisects the telecentric lens/camera assembly 40 along line 18-18 in FIG. 6. The system includes an imaging chamber 42 into which the plated culture dish is received and a support region 38 that supports the plated culture dish during imaging.

FIG. 19 is a magnified view of the imaging chamber 42 of the apparatus illustrated in FIG. 18. In the illustrated aspect, there are three light sources: top (50a), grazing (50b) and bottom (50c) light sources. Each light source as illustrated has twelve LED strips in a circular configuration (only a portion of that circle is illustrated in the cut away view of FIG. 19). With top and grazing light (50a, 50b) a black background is positioned underneath the plated culture dish (not shown). For the bottom light source to illuminate the plated culture dish, this background is moved out of the imaging chamber 42. There are three light diffusers installed provided for each illumination source: top (51a), grazing (51b) and bottom (51c).

The diffuser 51b for the grazing light source strips is attached to a lifting mechanism. As illustrated, the grazing light diffuser 51b is lifted out of the way by the lifting mechanism 51d for the indexing disc 251 to advance the plated culture dish to and from the imaging position.

Referring to FIG. 20, a mirror 33 is placed above the transparent cover 45 that permits illumination from the light sources 50c positioned below the transparent cover 45. The moveable black background 46 is placed beneath the transparent cover 45.

FIG. 20 is a top down perspective view of the structure that receives the plated culture dish (not shown) in the imaging station 253 of the image capture system 30. FIG. 20 also illustrates the mirror 33 adjacent to where the plated culture dish is to be placed by the indexing disc 279. As illustrated, the mirror 33 is placed such that the entire mirror 33 is beneath the bottom of the plated culture dish. Laterally, at least a portion of the mirror extends into a perimeter defined by the plated culture dish. However, as illustrated in FIG. 4, most of the mirror is outside the perimeter defined by the plated culture dish.

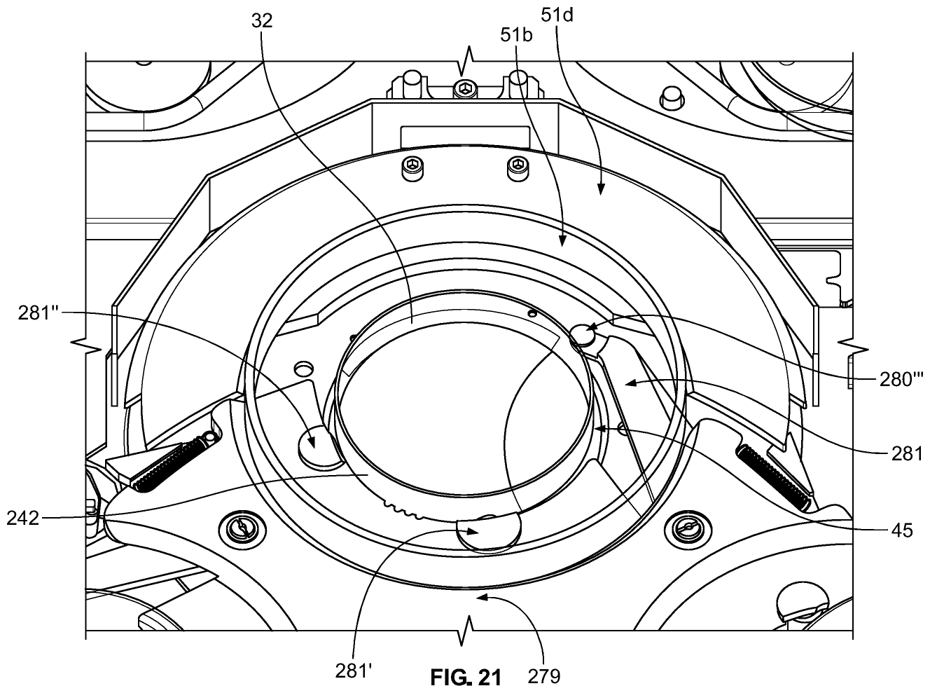

Referring to FIG. 21, the plated culture dish 242 is carried by an indexing disc 279. The indexing disc 279 is provided with plate bumpers 280′, 280″ and 280″. Bumper 280″' is mounted on a hinged arm 281 that is in an open position when the plated culture dish 242 is received by the indexing disc 279 from the conveyor 240. After the scan lift 244′ orients the plated culture dish 242 such that the label 32 is within the predetermined range and releases it to the indexing disc, the hinged arm 281 moves to the closed position to hold the plated culture dish 242 in place so that, when the indexing disc 279 advances the plated culture dish 242 to the imaging station 253, the label is aligned with the mirror 33. Grazing light diffuser 51b is also illustrated in FIG. 21.

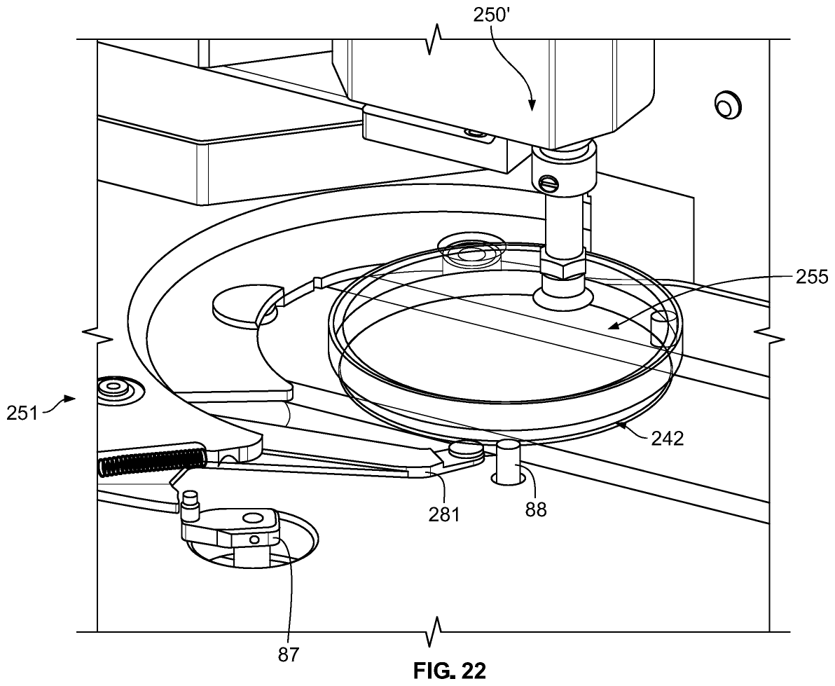

FIG. 22 illustrates how the plated culture dish 242 is advanced to the indexing disc exit position. As noted above, the upstream slot of the indexing disk contains the next plated culture dish so that imaging of the next plate can begin almost seamlessly with the exit of the previous plated culture dish from the imaging station. At the indexing disk 251 exit postion, a finger mechanism 87 opens the flipper 281, so that the plated culture dish 242 is conveyed to the stoppers 88 where the plated culture dish is held so that the lid 255 for the plated culture dish 242 can be placed on the plated culture dish using lid manipulator 250′. When the vacuum sensor (not shown) confirms the release of the lid from the suction cup 252′, the stoppers 88 are lowered and the plated culture dish is released.

FIG. 23 illustrates the return of the plated culture dish 242 to the entrance subsystem 275, where the plated culture dishes 242 ingress 276 into and egress 277 from the image capture module 200. The plate is stopped by the stoppers 90. A suction cup 243′ fixates the plate to a scan lift 244″. After confirmation of the vacuum, the scan lift 244″ is raised. The plated culture dish 242 is rotated and the barcode is scanned by scanner 259 to confirm the correct plate. Also, the plated culture dish 242 is oriented using the barcode and an offset setting. This way, if the plated culture dish 242 is called for a new cycle, the orientation is pre-defined for optimal throughput.

Optionally, label detection uses certain information stored by the system (referred to as a priori (mechanical) knowledge herein. Such information includes, but is not limited to information obtained from system calibration or known mechanical constants for system components. The information that the system and method has stored includes the surface area of the glass plate 300 illustrated in FIG. 15. When the glass plate is illuminated from the bottom, the visible part of the glass plate area is approximated by a circle 319.

The stored information also includes the mirror arc description. FIG. 15 illustrates the glass plate area 300 without a plated culture dish thereon as seen by bottom illumination (transmitted light). During calibration, the glass plate area 300 is approximated and defined by a circle 319, having a center 310. The center and radius of the circle is also approximated. The arc-shaped mirror 313 is identified during calibration. Both end locations 332, 334 of the arc-shaped mirror 313 are identified in relation to the glass plate area 300. The internal diameter of the arc-shaped mirror 313 is slightly less than the diameter of the glass plate 319 while the external diameter of the arc-shaped mirror 313 is larger than the diameter of the support plate 319. The glass plate 300 and the arc-shaped mirror 313 have a common center. The angle θ is the angle subtended by the arc-shaped mirror 313.

As explained above, the indexing disc 251 fixes the position of the culture dish relative to the imaging apparatus using bumpers 280′, 280″ and 280″′ and the flipper 281.

The mirror arc is bounded by lines 331 and 332 which intersect at the center 310 mentioned above. The angle θ is used to locate the angle of the end of the mirror relative to the support center 310. The perimeter of the support 319 is calculated from the support center 310 and the radius of the support.

To capture the image of the label, the label and the mirror are aligned. The orientation of the plated culture dish is determined by detecting the edge of the label and rotating the plated culture dish such that the label placement aligns with the mirror placement, ensuring that the label is reflected by the mirror. Referring to FIG. 6 the plated culture dish 11 is conveyed into the imaging apparatus by a conveyor system 240. A label (not shown) on the plated culture dish 11 is scanned as the plated culture dish 11 is transported past the scanner 249. Sensing the label placement allows the indexing disc 251 to receive the plated culture dish with the label in a position that will allow the label to be in alignment with the mirror 13 when the plated culture dish 11 is advanced to the imaging position. The imaging position is illustrated in FIG. 19.

As described previously, the label can be used to orient and align a current image of a plated culture dish with a prior image of the plated culture dish. Both images are first translated by aligning the dish center in the first image with the dish center in the second image. The angle defined by the label edges and the dish center is then used to rotate one image relative to the other one. Using the image of the labels as fiducial information facilitates the alignment of pixel data between images of the same plated culture dish taken over time.

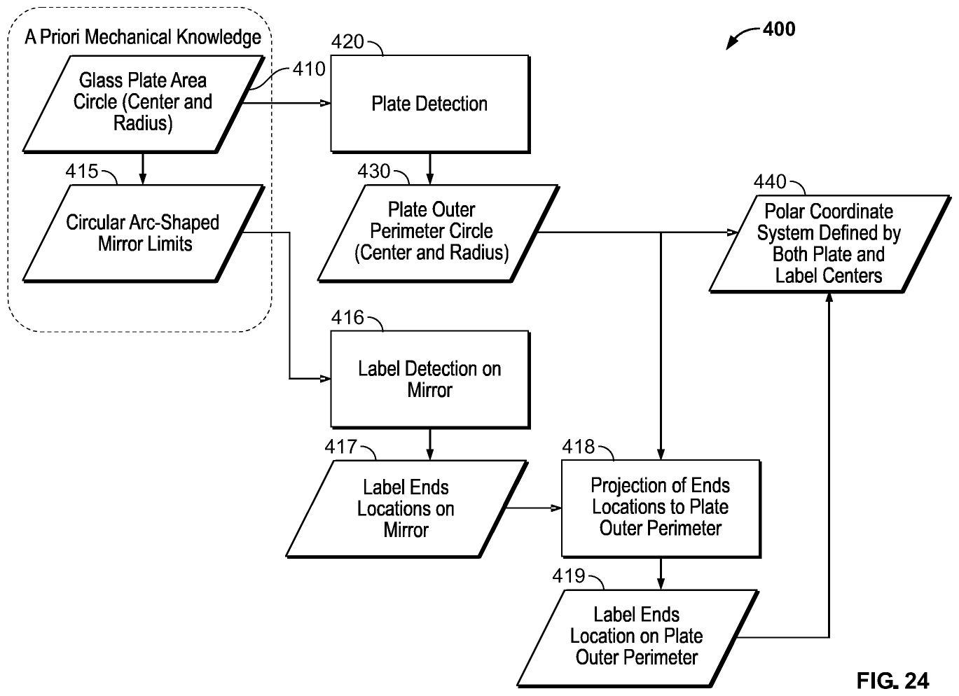

As described in detail herein, the system must detect the dish in order to obtain the information needed to understand the orientation of the plated culture dish not only for the current image, but for past and future images so that the images taken at different times can be aligned. In this way, pixels that change from image to image can be detected. The method by which the plate and label centers are determined are described in FIG. 24.

Once the dish centers are determined from dish detection, the image can be compared with a previous image of the same plated culture dish and the orientation of the plated culture dish in the imaging apparatus relative to the prior orientation of the plated culture dish is determined. Once both images are aligned with respect to their dish centers using translation, the images are then aligned using rotation with respect to the respective label centers (i.e., by aligning the label center in the first image with the dish center in the second image).

Optionally, the image capture system described herein may have a telecentric lens module that aligns and fixes the position of the telecentric lens and the camera of the imaging device with respect to the plated culture dish. The telecentric lens module comprises one or more brackets and one or more plates. Optionally, a ball joint may be used for tilting the telecentric lens with camera. This allows the axis of the telecentric lens and camera view to be set perpendicular to the plate surface (or at some other angle if desired).

Optionally, the image capture system described herein may be a module that is integrated with an incubator. U.S. Application Publication No. 2015/0299639 A1, which is hereby incorporated by reference in its entirety, discloses such an integrated incubator and image capture module that regulates the incubator atmosphere and obtains high-resolution digital images of sample specimens. In this instance, the image capture system may be in the form of a module that is an enclosed unit immediately adjacent to a sample incubator used to grow and maintain microbiological and cell cultures. This enables direct transport of the sample from the incubator into the environment of the image capture module with no transport through one or more intervening environments. Sample containers, such as dishes containing plated cultures, are conveyed into the image capture module through a port, or an ingress door of a port. Thereafter, a lid of the sample container may be removed such that an image capture unit may electronically image (e.g., digital photographs) the sample container. The lid may be replaced after the sample container has been imaged and the sample container may be conveyed back through the same door, or alternatively through an egress door of the port, for placement back into the controlled incubator environment to continue incubation. As noted in U.S. Application Publication No. 2015/0299639 A1, having the image capture module directly adjacent to the incubator reduces the amount of time the sample container is exposed to an external environment (with its lack of precisely controlled temperature and atmosphere and potential contaminants) while the sample container is imaged. Since the image capture module is enclosed, it acts as a shield between the lab atmosphere and the incubator atmosphere reducing the extent to which the lab atmosphere enters the incubator and the sample containers enter from the incubator and return thereto through the door.

As noted above, an image of the plated culture is obtained as described in the prior art. Such images are obtained using different exposure times. The exposure time is determined to provide a target intensity range in a region of interest of the image. In one instance of operating the system described herein, a color image is generated using side illumination only. The exposure time of the image is controlled so that the intensity range of the image obtained from the mirror arc is within the target intensity range. Once the image of the mirror with the target intensity range is obtained, a greyscale image is then obtained by keeping an image obtained using a single-color channel of the image. Typically, the most intense color channel is used to generate the grey scale image.

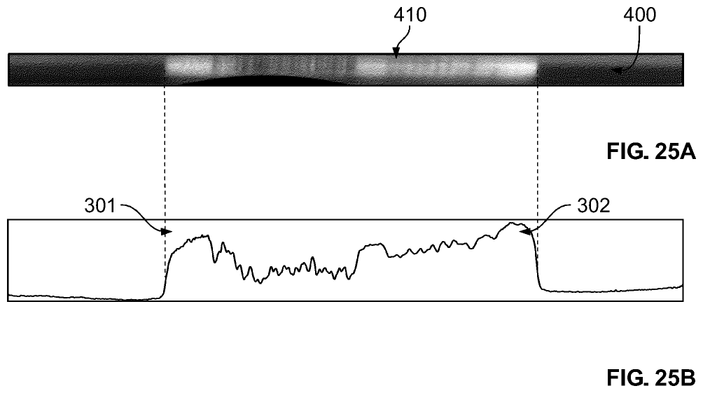

Referring to FIG. 25A, illustrated is a linear image of the mirror 400 with a reflection of the label 410. To obtain an angular profile along the mirror, the center 310 of the circle that defines the perimeter of the glass plate is used to define the origin of a polar coordinate system. As illustrated in FIG. 26A, the center 310 is also the center of the arc of the mirror 313. Here, the region of interest is the mirror 313 (FIG. 26A). Using the a priori knowledge described above regarding the mirror arc, the polar image of the mirror and the position of the label in the mirror is created.

FIG. 25B is a one-dimensional signal of the two-dimensional image in FIG. 25A. This one-dimensional image can be obtained in a variety of ways well known to one skilled in the art. For example, the mean for the columns in the polar coordinate image of FIG. 25A can used in a reduction operation like sum, max or min for all columns of the pixels in the FIG. 25A image. As illustrated in FIG. 25B, the two-dimensional polar image of the mirror is reduced along the radial dimension to the one-dimensional angular profile of the mirror and the position of the label relative to the ends of the mirror. Typically, the mean of all columns of the polar image are used to generate the angular profile.

Once the angular profile of the mirror is known, the reflection or image of the label on the mirror is detected. As described above with reference to FIG. 10, the label's reflection on the mirror is delimited by its lateral ends. In angular terms, the label lies between θₛₜₐᵣₜ and θ_{end}, which is illustrated in FIG. 25B as start and end angles 301, 302 along the angular profile.

In order to detect label on the mirror, Ω is used as a set of all possible pairs of angles (θₛₜₐᵣₜ,θ_{end}) on the mirror arc. The set is populated based on one (or more) physical lengths of the expected labels, with provisions for variations in label lengths. Although this allows for labels that are slightly longer than the specified length, it mostly allows for labels that are slightly shorter than the specified length. When the tolerances are subtracted from the label length, the system can identify coordinates (i.e., the ends) of crooked labels on the dish wall that make the label projection on the mirror shorter than the actual length of the mirror. In order to identify the ends of labels that are only partially reflected on the mirror arc, this set Ω can also include angle pairs spaced by less than the equivalent label length (up to 25% less than the label length). In such cases, one of the angles corresponds to one of the two mirror ends, since in these examples one end of the label extends beyond the edge of the mirror.

The detection of the label ends along the profile consists in maximizing a score function:

[symbol]

, [symbol] =arg max_{Ω}S_{label}(Ω) (1)

The score function is a combination of an edge-based term and a region-based term as follows:

S_{label}—αS_{edge}+βS_{region}, (2)

where α and β are weights. By using the intensity gradient (αS_{edge}) on the angular intensity profile, the first term favors local strong variations of intensity (the label ends 301, 302 illustrated in FIG. 25B). The second (βS_{region}) uses regional statistical information to identify the label region in the image information (i.e. a region that shares a mean intensity that is large relative to the rest of the profile). The first term uses the known label length and provides accuracy to the measurement because it clearly indicates label ends. It sums both intensity gradients at θₛₜₐᵣₜ and θ_{end}, therefore spaced by the known label lengths, which are part of the a priori knowledge described above. However, the first term is more sensitive to noise, (e.g. strong peaks inside the label reflection due to the barcode itself which can be seen by the variations in intensity along the label length in FIG. 25B). The second term therefore adds robustness by verifying that the label spans the distance between the label ends (or one label end and the end of the mirror). In other words, the second term ensures that variations in intensity along the label length are not interpreted as the end of a label.

The region-based contribution is the “Michelson” contrast of intensity I between regions of the mirror arc that are inside and outside the label. This is defined by the following:

$${S}_{r e g i o n} ( {θ}_{start} , {θ}_{e n d} ) = \frac{{I}_{i n} - {I}_{out}}{\overline{{I}_{i n}} + \overline{{I}_{out}}} \qquad ( 3 )$$

where Ī denotes the mean intensity in the region, e.g.,

$$\overline{{I}_{i n}} = \frac{1}{n} {Σ}_{θϵ [ {θ}_{start} , {θ}_{en ( d} ]} I ( θ )$$

wherein n is the number of points between θₛₜₐᵣₜ and θ_{end}.

The edge-based term is the contribution of the gradient magnitudes along the profile at both label ends. This is defined by the following relationship:

$${S}_{edge} ( {θ}_{s t art} , {θ}_{end} ) = \frac{\vert ∇ I ( {θ}_{start} ) \vert - \vert ∇ J ( {θ}_{end} ) \vert}{M} , \qquad ( 4 )$$

where ∇I(θ) denotes the gradient of intensity I at the angle point θ and M is the maximum intensity value used to generate the image as described above. For example, the maximum intensity M is 255 for 8 bits. However, if the gradient magnitudes of angle points θ are too close to the profile borders (i.e., to the mirror ends), they are not considered. This occurs most often (but not always) when angle pairs are describing labels that are only partially visible within the mirror arc (i.e., the entire image of the label is not in the mirror). This might also occur when the label is entirely reflected but has one lateral edge very close to the end of the mirror. The maximal contribution thus comes from a transition from dark to bright at θₛₜₐᵣₜ and backwards (i.e., from bright to dark) at θ_{end}. Note that minimum targets are expected from S_{edge} and S_{region} to consider the found region between ( [symbol] , [symbol] ) as a true label.

Referring to FIG. 26A, label end locations 312, 314 (128,129 in FIG. 5) on the dish contour 309 are finally deduced from ( [symbol] , [symbol] ) as the intersection points between the plated culture dish circle 309 and the lines passing through the label end locations 301, 302 on the mirror and the glass plate area center 310. For example, after dish detection, when the dish center is determined, the dish center 317 and the angular location (with respect to the dish center 317) of the label center 316 along the dish contour 309 is used to define a reference coordinate system to precisely identify the location of the image objects (e.g. colonies). As illustrated in FIG. 26A, using the label as fiducial, the coordinates of the object (r, θ) can be assigned relative to the fiducial. The next time the plate is received into the imaging apparatus, the apparatus will use the label and the plate center in the new image to identify the object. Image capture is described in detail in WO2015/114121, which is incorporated by reference herein.

FIG. 26B is an actual image of a culture dish obtained through bottom illumination. Overlaid on that image are the mirror ends 332, 334, the label ends 312, 314, the label center 316, the glass plate center 310 and the plated culture dish center 317. The fact that the centers 310 and 317 are not completely aligned is apparent. As stated elsewhere herein, a dish detection process must be performed to determine the dish center 317, the dish contour 309, etc.

The system and method herein deploy a method for detecting the dishes themselves in the image field of the imaging device having a telecentric lens. Dish detection leads to determining the dish center 317 and the dish contour 309. The dish contour and the dish center are then used to locate the ends of the label. The dish center is then used as the origin for the coordinate system used for image alignment and object detection. The coordinate system is also determined by the label center (which is determined by the reflection of the label in the mirror). As described above, the plated culture dish (or other receptacle being imaged) is placed on a larger glass plate 300 on which it is held along its circumference by a glass plate holder (127 in FIG. 5). When acquiring an image of a plated culture dish as described herein, the most external dish edge must be defined as precisely as possible since the integrity of the entire coordinate system described above depends upon assigning accurate coordinates in the coordinate system to the disc center. As described above, the outer perimeter of the culture dish is approximated by a circle, despite possible dish deformations caused by handling the dish (e.g. pushing on the dish wall can cause minor indentations).

All subsequent automatic inspection of the plates (e.g. growth detection, colony counting or identification) is restricted to this defined circular region. As noted above, the center of this region is the origin of the plate referential (i.e. the origin of the coordinate system described above). The coordinate system is used to precisely align the pixels of images taken at different times and to locate the colonies marked on the image and to be picked later (by a system such as IdentifA described above).

As with the label detection on the mirror described above, the definition of the dish perimeter requires specific a priori (mechanical) knowledge. Specifically, knowledge of the glass plate area 300, described as a circle, is required. The glass plate has an opaque glass plate holder (127), but it is only the portion of the glass plate that is illuminated (FIG. 15) that is used and approximated by a circle. Bottom illumination is used to define the limits of the visible part of the glass plate area approximated by 319 in FIG. 15.

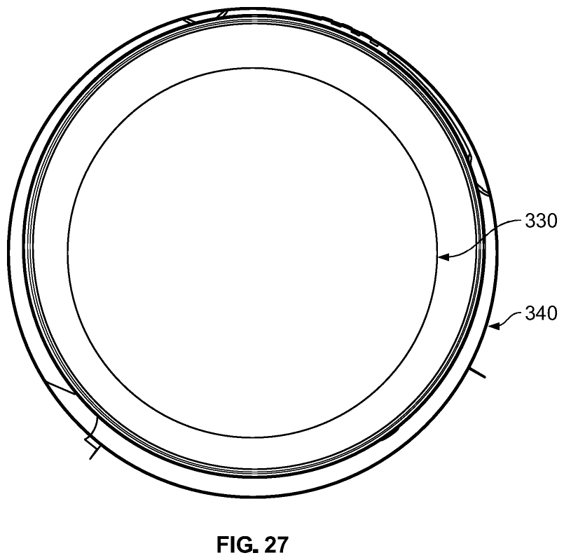

Referring to FIG. 27, 330 and 340 denote masks of the glass plate area beneath the culture dish (330) and extending beyond the culture dish (340), respectively. The inner mask 330 is a disc that shares its center with the center of the glass plate area 319 (FIG. 15). The image of the culture dish is obtained using bottom illumination. The region-of-interest 330 is dimensioned to fit within the circle defining a culture dish of any diameter that is accepted by the system. The region of interest 340 is a ring that will always be outside of the diameter of any plated culture dish accepted by the system. The outer region of interest 340 is used to measure a white statistic that describes the intensity of the bottom illumination transmitted only through the glass plate area (and not through both glass plate area and plate). Conversely, a black statistic is measured outside (beyond) the glass plate area circle 340. This is the opaque portion of the support plate holder 127.

The outer mask 340 is a ring 340 that also shares its center with the center if the glass plate area 319. The outer mask has an outer diameter equal to that of the glass plate area 319. The inner diameter of the outer mask 340 is dimensioned to be outside of a circle that defines a plated culture dish of any diameter that is accepted by the system.

As described above, using the image acquisition method described previously herein, a color image of the plated culture dish is obtained using conventional image capture. An image is obtained that provides a large intensity range within the circle 319 defined by the glass plate. The color channel that has the highest contrast between the mask regions 330 and 340 is retained and is used to generated a grayscale image. If the contrast between mask region 330 and mask region 340 is below a threshold experimentally adjusted using the least-absorbing dish (e.g. an empty dish without any media), this indicates that there is no dish in the system.

FIG. 24 is a flow chart for using the both plate center detection and label center detection to define a coordinate system that can be used to align images of the same plated culture dish obtained at different times. As describe above, a priori knowledge (step 410) is known by construction or found during calibration including a glass plate area circle (step 410) and the limits of the mirror (step 415) are used. In step 420, dish detection is applied to determine the outer perimeter of the circle (309) that delimits the dish in the system. In step 430, the plate outer circle is determined from the dish detection in step 420.

In step 416, an image of the mirror is obtained and from that image the label end locations 301, 302 on the mirror are detected. The label end locations in step 417 are therefore determined in step 416. In step 418, the label end locations on the mirror (301, 302 in FIG. 26A) are then projected back on the dish outer perimeter (step 430) resulting in label end locations on the plate perimeter (the data in step 419, which are 312, 314 in FIG. 26A). Step 430 determines 309 in FIG. 26A. From this, the center of the label 316 along the dish outer perimeter circle 309 is obtained from 128, 129 as previously described.

The reference system for the polar coordinates is defined using the dish perimeter center and the label center location determined in step 440. This is illustrated in FIGS. 26A and 26B. This reference system is used to align the pixels of images taken at different times and to locate the colonies marked on the image and to be picked later.

The dish perimeter circle 309 defines a region-of-interest (ROI) to which all subsequent automatic imaging and detection of the plates (e.g., growth detection, colony counting or identification) are restricted.

The median intensity value within the mask region 340 is computed (zero values of 340 corresponding to strict opaque regions are not included in the median computation). As noted above, the mask region 340 is the region outside the plate region of interest which includes those portions of the glass plate not covered by the plate. A white statistic, which is the mode of all intensity value within this mask region that are greater than the median intensity value, is obtained. This white statistic can be used to determine the perimeter of the plated culture dish, since the intensity in the glass plate transitions to a different intensity at the interface between the dish edge and the support.

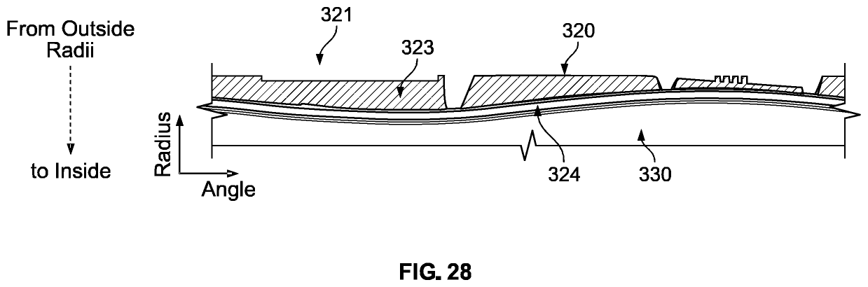

Referring to FIG. 28, illustrated is a polar image extending from the opaque outer portion of the glass dish support to inside the plated culture dish. The origin of the polar coordinate system is the center 310 of the circle that defines the perimeter of the glass plate. The inner outer limit of mask region 330 in FIG. 27 is less than the smallest plated culture dish accepted by the system and the glass plate perimeter 319 is the outer outer limit of the mask region 340 in FIG. 27. Therefore, FIG. 28 is a polar image that is obtained from the image in FIG. 27, i.e., through bottom illumination. The range of radii is from the outer perimeter of the glass plate 319 to the radii inside the plate perimeter 309 and based on the radius of the smallest plated culture dish accepted by the system to ensure that the inside of any plated culture dish deployed in the system will be reached. The radius range of the polar image is approximated to include radii around the whole circumference of the plate/support for both the opaque regions outside the glass plate circle 319 and regions inside the plated culture dish perimeter 309. Because FIG. 28 is a polar image, the radial dimensions from outside the glass plate area circle to inside the plate are for each column of pixels in the image. When the centers of the plated culture dish and the glass dish support are not perfectly aligned, the polar image reflects variation in the distance between the perimeter of the plated culture dish and the perimeter of the glass plate support. As noted above, the plate radius is determined by the range of plate radii accepted by the system. The white statistic is for region 340. A black statistic 321 is the intensity mean value beyond the glass plate area (i.e., for radii strictly greater than the glass plate area circle one).

To detect the plated culture dish, a strong transition from black (321) to white (323) intensities based on black and white statistics are determined first. As noted above, white statistics are calculated within the mask region 340 and are designed to always be inside the perimeter of the glass plate but outside the perimeter of the plated culture dish. These pixels are indicative of the glass dish support perimeter 322. This intensity transition is required to be greater than a predetermined threshold percentage (e.g. 70%) of the difference of the white statistic with the black statistic (which are mean values.

The width of region 323 is then determined by identifying pixels in a column with an intensity that remains strictly greater than a percentage of the white statistic (related to the minimal plate edge absorption and adjusted experimentally to 70%). This transition from the glass plate region to the plated culture dish perimeter is finally refined to be where the gradient from white to black is the greatest 324. From all of these transitions 324, the dish outer perimeter is approximated by a circle 309.

Although the system and method described herein has been described with reference to particular examples, it is to be understood that these examples are merely illustrative of the principles and applications of what is described and claimed. It is therefore to be understood that these and various other omissions, additions, and numerous modifications may be made to the illustrative examples and that other arrangements may be devised without departing from the spirit and scope of the appended claims.

1. A system for capturing an image of a plated culture dish, comprising:
an imaging device having a camera with a telecentric lens adapted to capture an image of a plated culture dish;
an indexing disc that receives the plated culture dish, the indexing disc receiving the plated culture dish from a conveyor and rotating the plated culture dish into a field of view of the telecentric lens;
a mirror positioned adjacent to a support for the plated culture dish, the mirror adapted to provide a reflection of a label on a side of the plated culture dish within the field of view of the telecentric lens; and
at least one light system for illuminating the plated culture dish for image capture.

2. The system of claim 1, wherein the mirror is adjacent to the side of the plated culture dish, the plated culture dish having a bottom, wherein at least a portion of the mirror is placed such that at least a portion of the mirror extends at least partially beneath the bottom of the plated culture dish at the side of the plated culture dish or no portion of the mirror extends at least partially beneath the bottom of the plated culture dish at the side of the plated culture dish.

3. The system of claim 2, wherein at least a portion of the mirror extends outward beyond a perimeter of the plated culture dish.

4. The system of any of claim 1, wherein the plated culture dish has a diameter and where the system receives plated culture dishes of different diameters.

5. The system of claim 1, further comprising a telecentric lens module that is adapted to align and fix a position of the telecentric lens and the camera of the imaging device with respect to the plated culture dish.

6. The system of claim 1, wherein the at least one light system includes a light emitting diode (LED).

7. The system of claim 6, where the light system comprises three light sources.

8. The system of claim 7, wherein the light sources are a tip light source, a side light source and a bottom light source and wherein, optionally, each light source comprises a plurality of LEDs arranged in a circular configuration.

9. The system of claim 8, further comprising a diffuser for each light source.

10. The system of claim 9, wherein the diffuser for the side light source comprises a lifting mechanism that moves the diffuser vertically thereby permitting the plated culture dish to be moved into the field of view of the telecentric lens.

11. The system of claim 1, wherein the mirror is positioned above a transparent cover.

12. The system of claim 11, wherein a moveable opaque background is positioned beneath the transparent cover.

13. The system of claim 12, wherein the indexing disc comprises a plurality of bumpers that contact the plated culture dish when received by the indexing disc, the indexing disc optionally comprising a hinged arm that is in an open position to receive the plated culture dish into an indexing disc receptacle wherein the hinged are is moved to a closed position when the plated culture dish is received by the receptacle.

14. The system of claim 1, further comprising a conveyor that transports the plated culture dish from an ingress location to the indexing disc and from the indexing disc to an egress location.

15. The system of claim 14, wherein the ingress location comprises a culture dish lift comprising a platform that rises beneath a plated culture dish placed at the ingress location, wherein the culture dish lift optionally comprises a sensor that detect a presence of the plated culture dish on the platform and further optionally comprises a securement for the plated culture dish on the platform, wherein the securement is optionally a suction cup.

16. The system of claim 14, wherein the conveyor further comprises a buffer position that stops the plated culture dish from advancing into the indexing disc and optionally further comprises a scanning station positioned downstream of the buffer position, wherein a scanner at the scanning station reads the label on the plated culture dish and wherein the scanning station optionally comprises a scanning lift comprising a platform that rises beneath a plated culture dish placed at the scanning station and that rotates the plated culture dish to place the label to be read by the scanner, wherein the scanning lift further comprises a securement for the plated culture dish on the platform, wherein the securement is optionally a suction cup.

17. The system of claim 1, further comprising a lid manipulator that removes a lid from the plated culture dish prior to the plated culture dish being received into the indexing disc, wherein the lid manipulator optionally comprises a securement that attaches to a lid on the plated culture dish to remove the lid therefrom and optionally wherein the system comprises a second lid manipulator that receives the lid from the lid manipulator and wherein the second lid manipulator places the lid back on the plated culture dish.

18. The system of claim 14, wherein the egress location comprises a culture dish scanning lift comprising a platform that rises beneath a plated culture dish placed at the egress location and that rotates the plated culture dish to place the label in a position to be read by a scanner at the egress location, wherein the scanning lift further comprises a securement for the plated culture dish on the platform, wherein the securement is optionally a suction cup.

19. The system of claim 1, wherein the system is an image capture module integrated with an incubator.

20. The system of claim 19, wherein the image capture module is adjacent to the incubator outside of a controlled cabinet environment.

21. The system of claim 1, wherein the indexing disc has a plurality of receptacles each for receiving the plated culture dish; and wherein the indexing disc moves the plated culture dish from a location, where it is received by the indexing disc, to the imaging device and from the imaging device to an exit location from the indexing disc.

22. A method for obtaining an image of a plated culture dish, the method comprising:
providing an imaging system comprising a camera, a telecentric lens, a support for receiving a plated culture dish for imaging, and a mirror adjacent the support;
providing the plated culture dish with a label located on and attached to a side of the plated culture dish;
positioning the plated culture dish in the imaging system at an imaging position such that the label is reflected in the mirror; and
capturing an image of the plated culture dish along with a reflection of the label in the mirror.

23. The method of claim 22, further comprising determining an orientation of the plated culture dish in the imaging position by identifying a center of the image of the plated culture dish and the center of the label, from which a location of ends of the label and a location of ends of the mirror relative to the center of the image of the plated culture dish are determined.

24. The method of claim 22, further comprising aligning pixels in a first image of the plated culture dish obtained at a first time with pixels of a second image of the plated culture dish obtained at a second time using the center of the image of the plated culture dish identified in the first image and the center of the plated culture dish is identified in the second image, the location of ends of the label determined in the first image and the location of the label ends determined in the second image.

25. The method of claim 24, wherein an angular profile of the mirror is determined from a one dimensional image of a two-dimensional image of the reflection of the label in the mirror.

26. The method of claim 22, wherein at least a portion of the mirror is placed such that at least a portion of the mirror extends at least partially beneath a bottom of the plated culture dish at the side of the plated culture dish or no portion of the mirror extends at least partially beneath the bottom of the plated culture dish at the side of the plated culture dish.

27. The method of claim 25, further comprising determining ends of the labels from the angular profile.

28. The method of claim 27, further comprising assigning coordinates to an object on the plated culture dish relative to the label and the center of the plated culture dish.
